SEQUENCE LISTING (20 sequences)

SEQ ID NO: 1 - DNA, 21 bp, Mus musculus
gccaagaagacatccctgaag

SEQ ID NO: 2 - DNA, 21 bp, Mus musculus
tgtggcagatacagatcaagc

SEQ ID NO: 3 - DNA, 19 bp, Mus musculus
ggagggaacggtccacgat

SEQ ID NO: 4 - DNA, 18 bp, Mus musculus
gagtccgcgtatccacaa

SEQ ID NO: 5 - DNA, 19 bp, Mus musculus
tttgtgcaagtggaacctg

SEQ ID NO: 6 - DNA, 20 bp, Mus musculus
tggactgctgtgccaaaata

SEQ ID NO: 7 - DNA, 21 bp, Mus musculus
tgatggcactgctgacatcat

SEQ ID NO: 8 - DNA, 20 bp, Mus musculus
tgtagcctttggaactgctt

SEQ ID NO: 9 - DNA, 20 bp, Mus musculus
tggtagctggaaatgtggtg

SEQ ID NO: 10 - DNA, 20 bp, Mus musculus
caggcacttctttgcactca

SEQ ID NO: 11 - DNA, 20 bp, Mus musculus
cgtgttcctacccccaatgt

SEQ ID NO: 12 - DNA, 24 bp, Mus musculus
tgtcatcatacttggcaggtttct

SEQ ID NO: 13 - DNA, 24 bp, Mus musculus
acggagatggatgtgccaaacgtc

SEQ ID NO: 14 - DNA, 23 bp, Mus musculus
cgagtaatccatttgcatgatgc

SEQ ID NO: 15 - DNA, 19 bp, Mus musculus
ttgacggaccccaaaagat

SEQ ID NO: 16 - DNA, 21 bp, Mus musculus
gaagctggatgctctcatctg

SEQ ID NO: 17 - DNA, 20 bp, Mus musculus
cagaagaatggaagagtcag

SEQ ID NO: 18 - DNA, 20 bp, Mus musculus
cagatatgcagggagtcacc

SEQ ID NO: 19 - DNA, 21 bp, Mus musculus
caactatagcgagctgcagga

SEQ ID NO: 20 - DNA, 22 bp, Mus musculus
acttggggtaccaggagagagt

(19) United States
(12) Patent Application Publication
(10) Pub. No.: US 2018/0303859 A1
(43) Pub. Date: Oct. 25, 2018

(54) Agent For Preventing And/Or Treating Scleroderma

(71) Applicant: THE UNIVERSITY OF TOKYO, Tokyo (JP)

(72) Inventors: Shinichi SATO, Tokyo (JP); Yoshihide ASANO, Tokyo (JP); Takashi YAMASHITA, Tokyo (JP)

(21) Appl. No.: 15/763,527

(22) PCT Filed: Sep. 26, 2016
(86) PCT No.: PCT/JP2016/078250

§ 371 Date: Mar. 27, 2018

(30) Foreign Application Priority Data
Sep. 30, 2015 (JP) 2015-192743

(51) Int. Cl. A61K 31/704; A61P 37/02

(52) U.S. Cl. CPC A61K 31/704; A61P 37/02

(57) ABSTRACT

The present invention aims to provide a prophylactic and/or therapeutic agent for scleroderma. The present invention also aims to provide a Th2 promotion inhibitor or a vascular stabilizing agent. Glycyrrhetinic acids and/or salts thereof are applied for prevention of scleroderma, treatment of scleroderma, inhibition of Th2 promotion, or stabilization of blood vessels.

### TECHNICAL FIELD

The present invention relates to a prophylactic and/or therapeutic agent for scleroderma, comprising glycyrrhetinic acids and/or salts thereof. The present invention also relates to a Th2 promotion inhibitor or a vascular stabilizing agent comprising glycyrrhetinic acids and/or salts thereof.

### BACKGROUND ART

Scleroderma is an autoimmune disease that affects multiple organs, and characteristically shows lesions in blood vessels, and fibrosis of the skin and various internal organs. The cause of scleroderma is still unclear, and therapeutic methods for its remission have not yet been established. At present, corticosteroids are commonly used for early-stage patients, and immunosuppressive agents are used for pulmonary symptoms. On the other hand, although consideration should also be given to suppression of accumulation of extracellular matrices such as collagen, which is a pathological condition caused by activation of fibroblasts in the skin and the like, no drug having such an effect has been discovered.

Glycyrrhetinic acids such as glycyrrhizic acid (glycyrrhizinic acid) are major pharmacologically active components of glycyrrhiza, which has been used in traditional Chinese medicine from ancient times. In Japan, those components have been developed as antiallergic agents, and therapeutic agents for hepatic fibrosis (Non-patent Document 1) and the like. Thus, their effectiveness and safety have been widely recognized.

### PRIOR ART DOCUMENT

### Non-Patent Documents

[Non-patent Document 1] Life Sciences, Volume 83, Issues 15-16, 2008, Pages 531-539

### SUMMARY OF THE INVENTION

### Technical Problem

An object of the present invention is to provide a prophylactic and/or therapeutic agent for scleroderma, whose cause is still unknown. Another object of the present invention is to provide a Th2 promotion inhibitor and a vascular stabilizing agent.

### Solution to Problem

The present inventors intensively studied to solve the problems described above. As a result, the present inventors discovered the fact that glycyrrhetinic acids such as glycyrrhizic acid, and salts thereof are effective for treatment of scleroderma, and the fact that they have a Th2 promotion-inhibiting action and a vascular stabilizing action, thereby completing the present invention.

That is, the present invention provides the following.

[1] A prophylactic and/or therapeutic agent for scleroderma, comprising glycyrrhetinic acids and/or salts thereof.

[2] The prophylactic and/or therapeutic agent for scleroderma according to [1], wherein the glycyrrhetinic acids comprise glycyrrhizic acid.

[3] The prophylactic and/or therapeutic agent for scleroderma according to [1] or [2], wherein the salts of the glycyrrhetinic acids comprise an ammonium salt or an alkali metal salt.

[4] The prophylactic and/or therapeutic agent for scleroderma according to any one of [1] to [3], wherein the scleroderma comprises systemic scleroderma.

[5] A Th2 promotion inhibitor comprising glycyrrhetinic acids and/or salts thereof.

[6] A vascular stabilizing agent comprising glycyrrhetinic acids and/or salts thereof.

[7] A method for prevention and/or treatment of scleroderma, comprising administering glycyrrhetinic acids and/or salts thereof to a subject.

[8] A method for inhibition of Th2 promotion, comprising administering glycyrrhetinic acids and/or salts thereof to a subject.

[9] A method for stabilizing blood vessels, comprising administering glycyrrhetinic acids and/or salts thereof to a subject.

### Advantageous Effect of the Invention

Glycyrrhetinic acids and salts thereof inhibit the promoted state of Th2, ameliorate inflammatory reaction and fibrosis, stabilize blood vessels, and prevent or treat scleroderma.

### BRIEF DESCRIPTION OF THE DRAWINGS

FIG. 1A shows a photograph showing the result of measurement of changes in the dermal thickness caused by administration of glycyrrhizic acid to bleomycin (BLM)-induced scleroderma model mice. FIG. 1B shows a graph showing the measurement results obtained by the measurement of the dermal thickness in FIG. 1A. FIG. 1C shows a graph showing changes in the amount of collagen caused by administration of glycyrrhizic acid to BLM-induced scleroderma model mice. Each amount of collagen is expressed as a relative value with respect to the amount of collagen in a group in which a solvent was administered instead of glycyrrhizic acid to control mice without BLM induction, which is taken as 1.

FIG. 2A shows photographs showing results of immunostaining of the skin of BLM-induced scleroderma model mice, which immunostaining was carried out for α-smooth muscle actin (α-SMA). FIG. 2B shows a graph showing the numbers of fibroblasts that were found to be positive for the α-SMA staining in ×400 viewing areas in FIG. 2A.

FIG. 3A shows results of flow cytometric evaluation of expression of IFN-γ, IL-4, and IL17A in CD4-positive cells collected from lymph nodes of BLM-induced scleroderma model mice. FIG. 3B shows graphs prepared from the results in FIG. 3A.

FIG. 4A shows results of flow cytometric evaluation of expression of IFN-γ, IL-4, and IL17A in CD4-positive cells collected from the spleen of BLM-induced scleroderma model mice. FIG. 4B shows graphs prepared from the results in FIG. 4A.

FIG. 5 shows photographs showing results of immunostaining of skin samples of BLM-induced scleroderma model mice using an anti-VE-cadherin antibody and an anti-fibroblast-specific protein 1 (FSP1) antibody. The top row shows results of staining with the anti-FSP1 antibody; the middle row shows results of staining with the VE-cadherin antibody; and the bottom row shows results obtained by merging the results shown in the top row and the middle row. The left column shows results from a control group without BLM induction; the middle column shows results from a group in which a solvent was administered to BLM-induced scleroderma model mice; and the right columns shows results from a group in which glycyrrhizic acid was added to BLM-induced scleroderma model mice. The arrows in the bottom row indicate cells stained for both VE-cadherin and FSP1.

FIG. 6A shows photographs showing results obtained by administering a solvent or glycyrrhizic acid, and then Evans blue dye, to mice deficient for vascular endothelial cell-specific Fli1, dissecting the mice, and then observing cutaneous blood vessels from the dermal side. FIG. 6B shows a graph showing results of measurement of extravasation of Evans blue dye. Each amount of extravasation is expressed as a relative value with respect to the amount of extravasation in a group in which a solvent was administered instead of glycyrrhizic acid to control mice without BLM induction, which is taken as 1.

### DETAILED DESCRIPTION

Embodiments of the present invention are described below.

The present invention relates to a prophylactic and/or therapeutic agent for scleroderma, comprising glycyrrhetinic acids and/or salts thereof. The present invention also relates to a Th2 promotion inhibitor or a vascular stabilizing agent comprising glycyrrhetinic acids and/or salts thereof.

(Prophylactic and/or Therapeutic Agent for Scleroderma)

Scleroderma is a disease whose main symptom is a skin disorder that causes hardening of the skin. Scleroderma can be largely classified into systemic scleroderma, which is characterized in hardening (sclerosis) of the skin and internal organs, and which has a chronic course, and localized scleroderma, in which sclerosis occurs only in the skin.

Systemic scleroderma is thought to be a disease which mainly causes the following three abnormalities: autoimmunity, fibrosis, and angiopathy. The autoimmunity results in positivity of autoantibodies such as anti-centromere antibodies, anti-topoisomerase I (Scl-70) antibodies, anti-U1 RNP antibodies, and anti-RNA polymerase antibodies. Since these autoantibodies are thought to appear in blood before the occurrence of the symptoms, the agent of the present invention may be applied for preventive purposes to patients who are positive for these autoantibodies even when the patients do not show the symptoms.

The fibrosis occurs due to excessive accumulation of collagen fibers in the skin caused by active production of collagen fibers by fibroblasts. This excessive production of collagen fibers is thought to occur due to production of various cell growth factors and substances called cytokines by lymphocytes in the skin followed by their complex interactions that lead to stimulation of fibroblasts.

An example of the angiopathy is Raynaud's phenomenon, which is a disorder found in not less than 80% of patients with systemic scleroderma. The Raynaud's phenomenon is vasospasm that occurs in various portions of hands in response to coldness or emotional stress, and causes reversible discomfort and color changes (pallor, cyanosis, erythema, or a combination of any of these) in fingers. When the fibrosis occurs also in blood vessels, hardening of the blood vessels occurs, leading to a decrease in blood flow, which may result in ulceration. Such vascular sclerosis and a decrease in blood flow occur not only in the skin, but also in internal organs. For example, when they occur in blood vessels of the lung, pulmonary hypertension occurs. When they occur in blood vessels of the kidney, symptoms called scleroderma renal crisis occurs. Patients with scleroderma show nail fold bleeding in some cases, and this is thought to be a symptom reflecting the angiopathy.

According to “Systemic Scleroderma—Diagnostic Criteria (2010)”, diagnosis of systemic scleroderma is made based on a major criterion and minor criteria, wherein the major criterion is dermal sclerosis in areas beyond fingers or toes, and the minor criteria are as follows: 1) dermal sclerosis localized to fingers or toes; 2) depressed scars at fingertips, or atrophy of finger pads; 3) bilateral fibrosis of the base of the lung; and 4) positivity of an anti-topoisomerase I (Scl-70) antibody, an anti-centromere antibody, or an anti-RNA polymerase III antibody. In cases where the major criterion, or both the minor criterion 1) and at least one of the minor criteria 2) to 4) is/are satisfied, a patient is diagnosed with systemic scleroderma.

Localized scleroderma is a disease in which fibrous sclerosis occurs in the skin and the subcutaneous adipose tissue as its base, and muscle and bone. This fibrous sclerosis is recognized as eruption on the skin. The eruption shows a large variation among patients. In some cases, dermal sclerosis can be obviously recognized, while in other cases, it is recognized as recessed areas. Regarding the color of the eruption, the eruption is often accompanied by erythema, or appears brown or white. Based on the shape of the eruption, localized scleroderma can be classified into morphea and linear scleroderma. As a special form of linear scleroderma, those occurring on the face or the head and neck area are called scleroderma en coup de sabre, and they reach the scalp to cause alopecia. Morphea can be classified into the localized form, the guttate form, and the generalized form based on the size and the number of eruptions.

In the present invention, the prevention of scleroderma means prevention of development of scleroderma. The prevention of scleroderma also includes inhibition or suppression of further exacerbation of scleroderma symptoms in various stages such as the early phase of the development. The treatment of scleroderma means amelioration, alleviation, or prevention or suppression of exacerbation of scleroderma symptoms.

(Th2 Promotion Inhibitor)

Th1 cells and Th2 cells are subsets of helper T cells. Th1 activates cellular immunity, and Th2 activates humoral immunity. Immune responses are regulated by the balance between these. Inappropriate regulation of the Th1/Th2 balance may lead to development of various diseases including autoimmune diseases. Autoimmune diseases can be largely classified into organ-specific autoimmune diseases such as Basedow's disease, and systemic (non-organ-specific) autoimmune diseases such as scleroderma. In general, promotion of Th1 induces organ-specific autoimmune diseases, and promotion of Th2 induces systemic autoimmune diseases. The Th2 promotion inhibitor in the present invention suppresses such a promoted state of Th2. Thus, the Th2 promotion inhibitor in the present invention is applicable to systemic autoimmune diseases such as scleroderma, systemic lupus erythematosus, rheumatoid arthritis, polymyositis, dermatomyositis, Sjogren's syndrome, mixed connective tissue disease, antiphospholipid antibody syndrome, microscopic polyangiitis, and granulomatosis with polyangiitis.

(Vascular Stabilizing Agent)

The vascular stabilization in the present invention means amelioration of vascular fragility and/or a state where vascular permeability is promoted, and/or amelioration of extravasation. Thus, the vascular stabilizing agent in the present invention is applicable to diseases that exhibit vascular fragility, promoted vascular permeability, and/or extravasation symptoms, and examples of such diseases include vascular leak syndrome; Ehlers-Danlos syndrome; purpura such as Schoenlein-Henoch purpura or purpura simplex; and hereditary hemorrhagic telangiectasia (Rendu-Osler-Weber disease). Further, the vascular stabilizing agent may be applied to a patient before administration of an anticancer drug, for prevention of extravasation of the anticancer drug.

(Glycyrrhetinic Acids and/or Salts Thereof)

The glycyrrhetinic acids in the present invention mean those having a pentacyclic triterpene structure. Examples of the glycyrrhetinic acids include, but are not limited to, glycyrrhizic acid, glycyrrhetinic acid, 18α-glycyrrhetinic acid 3-O-glucuronide, methyl glycyrrhetinate, stearyl glycyrrhetinate, pyridoxine glycyrrhetinate, glyceryl glycyrrhetinate, glycyrrhetinyl stearate, and carbenoxolone. These may be used individually, or two or more of these may be used in combination. Among these glycyrrhetinic acids, glycyrrhizic acid is preferred. The glycyrrhetinic acids in the present invention may be a crude extract from glycyrrhiza, which is a legume, as long as it contains glycyrrhetinic acids.

The salts of glycyrrhetinic acids in the present invention are not limited as long as they are pharmaceutically acceptable. Examples of the salts of glycyrrhetinic acids include salts of alkali metals (potassium, sodium, and the like), salts of alkaline earth metals (calcium, magnesium, and the like), ammonium salts, and salts of pharmaceutically acceptable organic amines (tetramethylammonium, triethylammonium, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine, and the like). In particular, ammonium salts and alkali metal salts are preferred. Disodium glycyrrhizinate, dipotassium glycyrrhizinate, and monoammonium glycyrrhizinate are more preferred. These may be used individually, or two or more of these may be used in combination. The definition of these salts may also be applied to the fatty acid esters of glycyrrhizic acid (for example, stearyl glycyrrhizinate and glyceryl glycyrrhizinate).

The agent in the present invention may be used alone, or in combination with a known prophylactic/therapeutic agent(s) for scleroderma, Th2 promotion inhibitor(s), and/or vascular stabilizing agent(s). By the combined use, enhancement of the prophylactic/therapeutic effect can be expected. The prophylactic and/or therapeutic agent(s) to be used in combination may be included as a component(s) of the agent in the present invention, or may be formulated separately from the agent of the present invention and placed in combination with the agent of the present invention to provide a kit that allows combined use of these agents, as long as the effect of the agent in the present invention is not reduced or lost.

Regarding the formulation, in addition to the effective component(s) described above, secondary components such as excipients, lubricants, disintegrators, binders, stabilizers, surfactants, diluents, additives, lubricating agents, antiseptics, and coating agents may be included, if necessary, to provide a pharmaceutical composition as long as the effect of the present invention is not deteriorated. A high-concentration solution containing glycyrrhetinic acids and/or salts thereof may undergo gelation at a low pH in the stomach or the like. For prevention of the gelation, addition of a phosphoric acid salt capable of increasing the pH (for example, disodium hydrogen phosphate or potassium dihydrogen phosphate), or L-arginine, which has an action to prevent gelation, is effective. The dosage form of the agent in the present invention is not limited, and may be appropriately selected depending on the usage. Specific examples of the formulation include tablets, balls, powders, liquids, granules, capsules, syrups, gels, and decoctions.

The dose of the glycyrrhetinic acids and/or salts thereof in the present invention is not limited as long as it allows production of a pharmacological effect, and may vary depending on the symptoms, age, and the like. The dose per administration is preferably 50 to 150 mg, more preferably 70 to 100 mg, still more preferably 80 mg. The administration may be carried out once to several times per day. The mode of administration is not limited, and examples of the mode of administration include oral administration, sublingual administration, intravenous administration, subcutaneous administration, transdermal administration, and intraperitoneal administration. The subject to which the agent is to be administered may be any animal including human, mouse, rat, monkey, rabbit, and guinea pig. Human is especially preferred.

### EXAMPLES

The present invention is described below more concretely by way of Examples. However, the present invention is not limited to these Examples as long as the spirit of the present invention is not spoiled.

### <Example 1> Study on Fibrosis in Scleroderma

To the skin of the back of 8-week-old C57BL/6 mice (wild type), 1 mg/ml bleomycin (BLM) solution prepared with phosphate buffered saline (PBS) was intracutaneously injected at a dose of 300 μg every day for four weeks to prepare BLM-induced scleroderma model mice. A control group was provided by subcutaneous injection of the same amount of PBS. At the same time, monoammonium glycyrrhizinate (Cokey Co., Ltd.) dissolved in PBS was intraperitoneally administered at a dose of 30 mg/kg every day for four weeks. A control group was provided by intraperitoneal administration of the same amount of PBS. Using a skin biopsy punch with a diameter of 6 mm, the skin tissue at the injection site was collected, and subjected to Hematoxylin & Eosin staining to measure the dermal thickness. Further, a skin sample collected by the same method was subjected to quantification of the collagen content using a QuickZyme Total Collagen Assay kit (QuickZyme BioSciences B.V., Netherlands).

Paraffin sections were prepared using the skin of the back at the injection site of each BLM-induced scleroderma model mouse, and reacted with an anti-α-smooth muscle actin (α-SMA) antibody (Sigma-Aldrich, St. Louis, Mo., USA) according to the manufacturer's instruction provided for the VEVTOR M.O.M Immunodetection Kit (Vector laboratories, Burlingame, Calif., USA), followed by coloring with DAB (0.2 mg/ml, DOJINDO LABORATORIES, Kumamoto, JAPAN). For each mouse skin sample, three photographs of the superficial dermal layer were randomly taken for ×400 viewing areas, and the numbers of spindle-shaped cells positive for α-SMA were counted. Their average was regarded as the number of α-SMA-positive fibroblasts in the mouse. Comparison was made between the glycyrrhizic acid group and the control group.

Further, RNA was extracted from the skin of the back at the injection site of each BLM-induced scleroderma model mouse using an RNeasy min kit (Qiagen Valencia, Calif., USA), and then reverse-transcribed into cDNA using iScript cDNA Synthesis Kits (Bio-Rad, Hercules, Calif., USA). Measurement by quantitative real-time PCR was carried out using a SYBR Green PCR Master Mix (Life technologies) with ABI prism 7000 (Life technologies). The measurement was carried out in triplicate for each sample to calculate the average value. As a reference gene, Gapdh was used. The relative expression level of mRNA of the subject gene was calculated by the ^{ΔΔ}Ct method. Primers having the following sequences were used.

Col1a1
(SEQ ID NO: 1)
F: GCCAAGAAGACATCCCTGAAG
(SEQ ID NO: 2)
R: TGTGGCAGATACAGATCAAGC
Col1a2
(SEQ ID NO: 3)
F: GGAGGGAACGGTCCACGAT
(SEQ ID NO: 4)
R: GAGTCCGCGTATCCACAA
Col3a1
(SEQ ID NO: 5)
F: TTTGTGCAAGTGGAACCTG
(SEQ ID NO: 6)
R: TGGACTGCTGTGCCAAAATA
Mmp13
(SEQ ID NO: 7)
F: TGATGGCACTGCTGACATCAT
(SEQ ID NO: 8)
R: TGTAGCCTTTGGAACTGCTT
Thbs1
(SEQ ID NO: 9)
F: TGGTAGCTGGAAATGTGGTG
(SEQ ID NO: 10)
R: CAGGCACTTCTTTGCACTCA
Gapdh
(SEQ ID NO: 11)
F: CGTGTTCCTACCCCCAATGT
(SEQ ID NO: 12)
R: TGTCATCATACTTGGCAGGTTTCT

<Results>

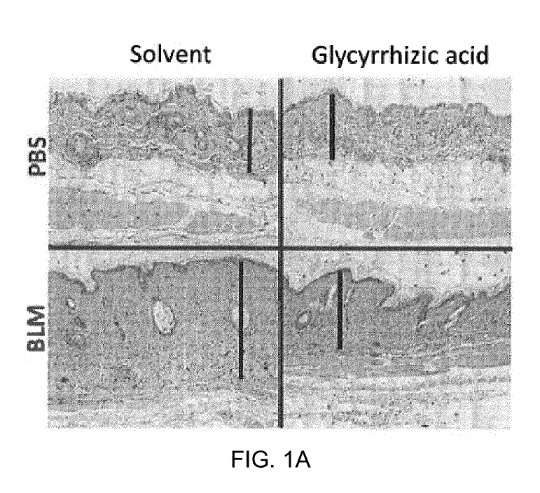

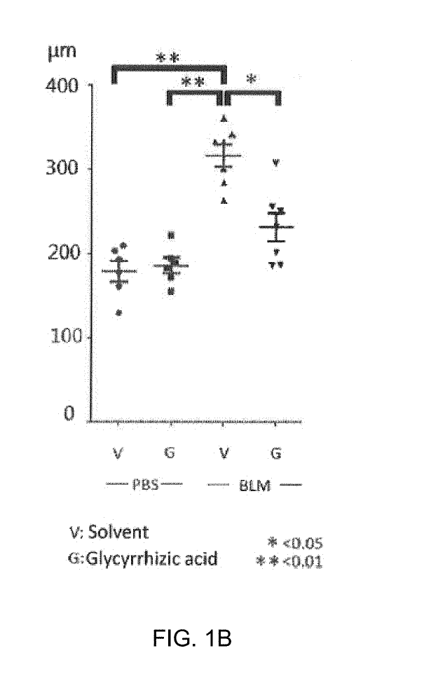

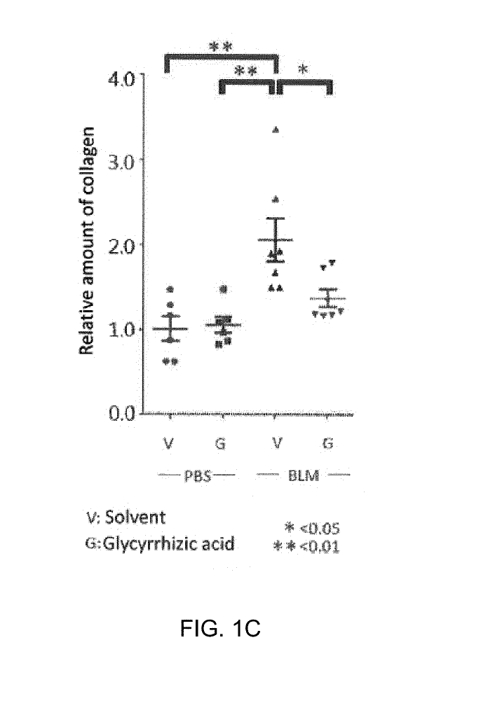

First, in order to study fibrosis in the BLM-induced scleroderma model mice, dermal thickness of the mice was measured. As a result, the model mice were found to have increased dermal thickness relative to the control mice to which PBS was administered instead of BLM (FIGS. 1, A and B). That is, induction of scleroderma with BLM caused an increase in the dermal thickness. As a result of the administration of glycyrrhizic acid to the model mice and the control mice, it was shown that the thickening of the dermis was reduced in the BLM-induced scleroderma model mice (FIGS. 1, A and B). On the other hand, in the control mice that were not subjected to BLM induction, the administration of glycyrrhizic acid did not cause any change in the dermal thickness. Thus, it was shown that glycyrrhizic acid does not act on normal dermis, and exerts the thickening-suppressing effect only on the thickened dermis of the scleroderma model mice.

The quantification of collagen fibers in the BLM-induced scleroderma model mice showed that the amount of collagen fibers, which had increased in the BLM-induced scleroderma model mice, was decreased by the administration of glycyrrhizic acid (FIG. 1, C). In the control mice that were not subjected to BLM induction, the administration of glycyrrhizic acid did not cause any change in the amount of collagen fibers. Thus, it was shown that glycyrrhizic acid does not act on collagen fibers in normal mice, and exerts its effect only on collagen fibers in the scleroderma model mice.

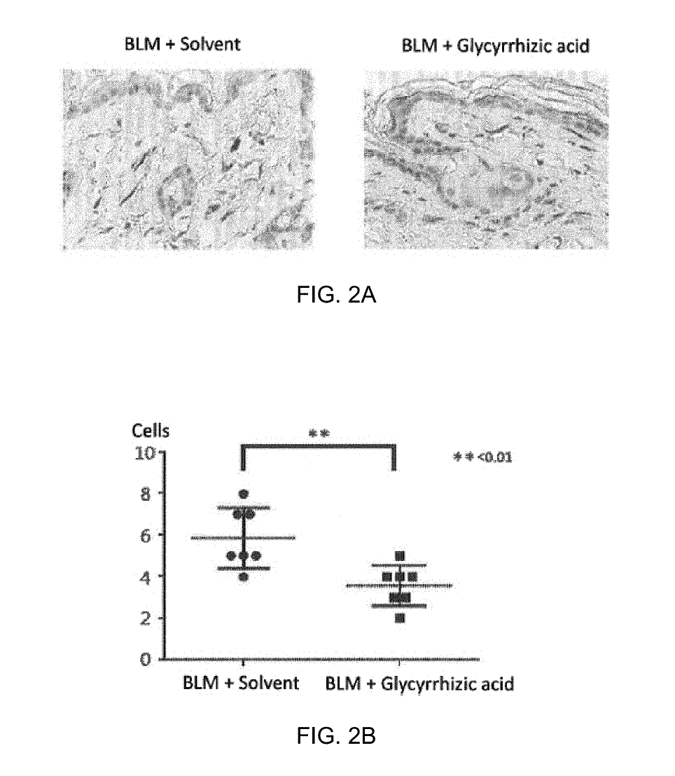

The administration of glycyrrhizic acid to the BLM-induced scleroderma model mice caused a significant decrease in the number of myofibroblasts in the skin (FIGS. 2, A and B). Since myofibroblasts are known to produce collagen fibers, it is thought that the decrease in myofibroblasts may have contributed to, as one of factors, the decrease in collagen fibers caused by the administration of glycyrrhizic acid in the scleroderma model mice.

Further, as a result of measurement of the mRNA levels of genes involved in production of collagen fibers in the scleroderma model mice, it was found that the administration of glycyrrhizic acid caused significant decreases in mRNAs of Col1a1, Col1a2, and Col3a1, which are collagen genes encoding collagen fibers, and a significant increase in mRNA of Mmp13, which is a gene encoding collagenase. Further, the administration of glycyrrhizic acid to the scleroderma model mice also caused a decrease in mRNA of thrombospondin (Thbs1), which converts latent TGF-β into active TGF-β. Active TGF-β is known to promote production of collagen fibers.

Thus, as a result of the study on fibrosis in the BLM-induced scleroderma model mice, it was found that administration of glycyrrhizic acid effectively reduces thickening of the dermis and the amount of collagen fibers. This effect was suggested to be due to a decrease in the number of myofibroblasts that produce collagen fibers, a decrease in the mRNA expression levels of collagen genes, an increase in the mRNA expression level of a collagenase gene, and a decrease in the mRNA expression level of thrombospondin.

### <Example 2> Study on Inflammation and Immune Abnormality in Scleroderma

BLM-induced scleroderma model mice were prepared by the same treatment as in Example 1 except that the bleomycin administration treatment was carried out for only one week. On the last day of the administration, bilateral inguinal lymph nodes and the spleen were removed from each mouse. After mashing the lymph nodes, lymphocytes were separated therefrom. To blood cells removed from the spleen, RBC lysis buffer (0.0017 M Trizma, Sigma-Aldrich, 0.1 M NH₄Cl 2.675 g, Sigma-Aldrich) was added to separate lymphocytes. The lymphocytes were subjected to staining of surface antigens using an anti-CD3 antibody (17A2), anti-CD4 antibody (RM4-5), and anti-CD8a antibody (53-6.7). Further, stimulation was carried out with 10 ng/ml phorbol myristate acetate (Sigma-Aldrich), 1 μg/ml ionomycin (Sigma-Aldrich), and 1 μg/ml brefeldin A (GolgiStop; BD PharMingen) for four hours, and staining of intracellular antigens was carried out using an anti-interleukin (IL)-4 antibody (11B11), anti-IL17A antibody (TC11-18HC0.1), and anti-interferon (IFN)-γ antibody (XMG1.2) (all antibodies were manufactured by BioLegend, San Diego, Calif., USA). The stained lymphocytes were analyzed using a FACS Verseflow cytometer (BD Biosciences).

Further, from the skin of the back at the injection site of each BLM-induced scleroderma model mouse, RNA was extracted by the same method as in Example 1, and the mRNA levels of IL4, IL1 b, and arginase were measured. Primers having the following sequences were used.

IL4
(SEQ ID NO: 13)
F: ACGGAGATGGATGTGCCAAACGTC
(SEQ ID NO: 14)
R: CGAGTAATCCATTTGCATGATGC
IL1b
(SEQ ID NO: 15)
F: TTGACGGACCCCAAAAGAT
(SEQ ID NO: 16)
R: GAAGCTGGATGCTCTCATCTG
Arg1
(SEQ ID NO: 17)
F: CAGAAGAATGGAAGAGTCAG
(SEQ ID NO: 18)
R: CAGATATGCAGGGAGTCACC

<Results>

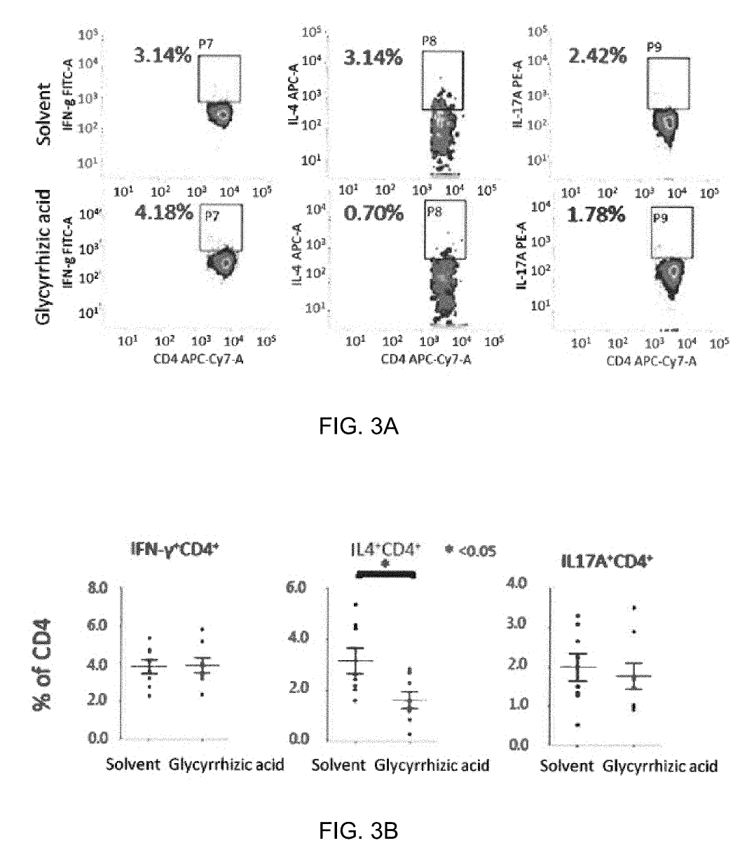

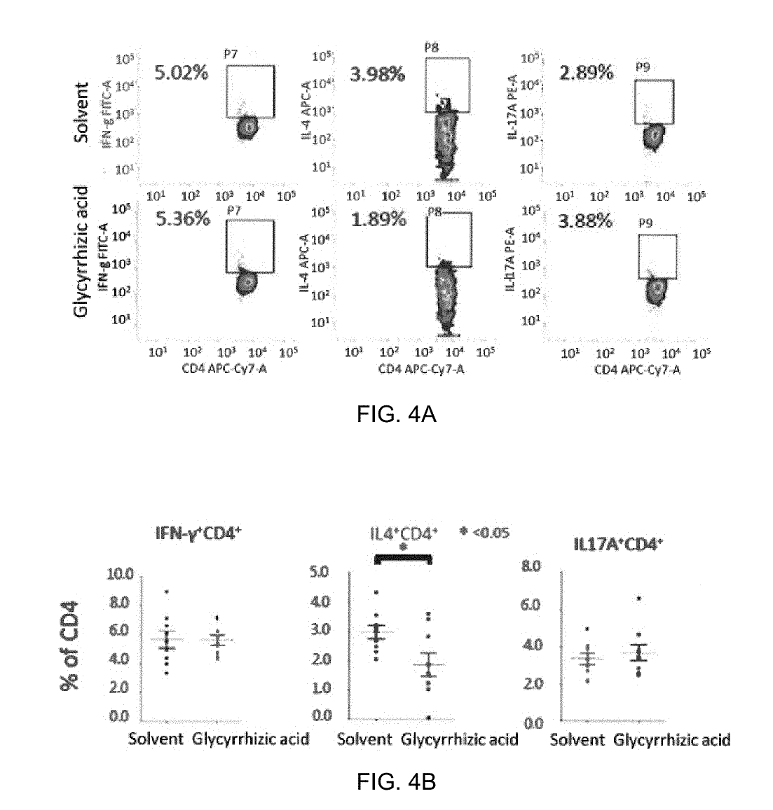

The ratio of IL4-positive cells among CD4-positive T cells in the lymph nodes or the spleen of each BLM-induced scleroderma model mouse was measured by FACS. As a result, significant decreases in IL4-positive cells due to the administration of glycyrrhizic acid were found in both the lymph nodes and the spleen (FIGS. 3 and 4). On the other hand, the administration of glycyrrhizic acid did not cause any change in the ratios of IFN-γ- or IL17A-positive cells among CD4-positive cells.

The influence of administration of glycyrrhizic acid on cytokine production in skin tissues of the BLM-induced scleroderma model mice was studied. As a result, the administration of glycyrrhizic acid decreased mRNAs of interleukin 4 and interleukin 1 b. Further, the administration of glycyrrhizic acid decreased mRNA of arginase, which is a marker for M2 macrophages.

Thus, as a result of the study on the influence of glycyrrhizic acid on inflammation and immune abnormality in the BLM-induced scleroderma model mice, IL4-positive CD4-positive T cells, which are Th2 cells, showed a significant decrease due to the administration of glycyrrhizic acid. On the other hand, IFN-γ-positive CD4-positive T cells, which are Th1 cells, and IL17A-positive CD4-positive T cells, which are Th17 cells, did not show remarkable changes caused by the administration of glycyrrhizic acid. Further, the administration of glycyrrhizic acid caused significant decreases in IL4, which induces differentiation into Th2 cells, and IL1 b, which is a proinflammatory cytokine, as well as a decrease in arginase, which is a marker for M2 macrophages induced by IL4. These results suggest the possibility that glycyrrhizic acid may suppress inflammation and ameliorate the Th2-dominant environment in the scleroderma model mice. Scleroderma is an autoimmune disease, and Th2 cells, which activate humoral immunity, are known to be dominant in patients with this disease. Glycyrrhizic acid was suggested to suppress such dominance of Th2.

### <Example 3> Study on Angiopathy in Scleroderma

In patients with scleroderma, vascular endothelial disorder is found in addition to fibrosis. This has been suggested to be due to transition of vascular endothelium to mesenchymal cells such as fibroblasts (endothelial-to-mesenchymal transition (Endo-MT)). In view of this, how Endo-MT is influenced by glycyrrhizic acid was studied using scleroderma model mice.

Paraffin sections were prepared using skin samples of the injection site of BLM-induced scleroderma model mice prepared under the same conditions as in Example 1. A rabbit anti-VE-cadherin antibody (Santa Cruz Biotechnolog) and a goat anti-fibroblast-specific protein 1 (FSP1) antibody (abcam, Cambridge, UK) as primary antibodies, and an FITC-conjugated donkey anti-rabbit IgG antibody (Santa Cruz Biotechnolog) and an Alexa Fluor donkey 555 anti-goat IgG antibody (Invitrogen, Carlsbad, Calif., USA) as secondary antibodies, were reacted with the sections, and nuclear staining was performed using Vectashield with DAPI (Vector Laboratories, Burlingame, Calif., USA). Observation was carried out using Bio Zero BZ-8000 (Keyence, Osaka, Japan) at wavelengths of 495 nm (green), 565 nm (red), and 400 nm (blue). The number of cells stained with both FSP1 and VE-Cadherin, which are observed as green color and red color, respectively (cells that had undergone Endo-MT), was compared. Further, from the skin samples, RNA was extracted by the same method as in Example 1, and the mRNA level of Snail1 was measured. Primers having the following sequences were used.

Snail1
F:
(SEQ ID NO: 19)
CAACTATAGCGAGCTGCAGGA
R:
(SEQ ID NO: 20)
ACTTGGGGTACCAGGAGAGAGT

Subsequently, under the same conditions as in Example 1, monoammonium glycyrrhizinate or PBS was intraperitoneally administered to 10-week-old mice deficient for vascular endothelial cell-specific Fli1 (Fli1 flox/flox; Tie2-Cre: see American Journal of Pathology, April 2010 Volume 176, Issue 4, p 1983-1998), which are model mice for angiopathy in scleroderma, or to control mice (Fli1 flox/flox). Two weeks later, 200 μl of Evans blue dye (0.5% in PBS) was administered into the tail vein of each mouse, and the mouse was euthanized 30 minutes thereafter, followed by incision of the skin from the middle of the abdomen. Cutaneous blood vessels were macroscopically observed from the dermal side to evaluate the degree of leakage of the dye. Further, for measurement of extravasation of Evans blue, skin was collected from three positions using a skin biopsy punch with a diameter of 4 mm, and then allowed to dissolve in formamide at 37° C. for 24 hours, followed by measurement of the absorbance at a wavelength of 620 nm using a microplate reader.

<Results>

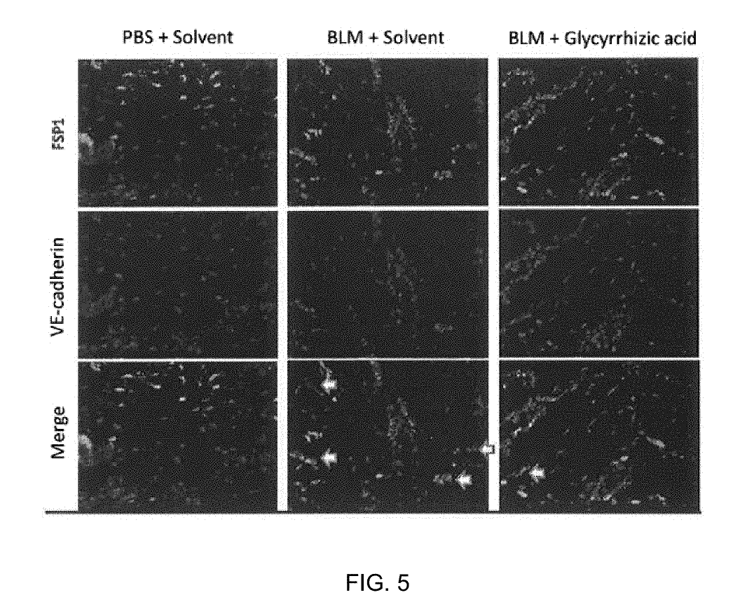

FIG. 5 shows results of the immunostaining using the anti-VE-cadherin antibody and the anti-FSP1 antibody. In FIG. 5, the arrows in the merged photographs in the bottom row indicate cells stained with both the anti-VE-cadherin antibody and the anti-FSP1 antibody, that is, cells that have undergone Endo-MT. The administration of bleomycin caused an increase in the cells that have undergone Endo-MT (BLM+solvent, the bottom row in FIG. 5), and this increasing action was suppressed by the administration of glycyrrhizic acid (BLM+glycyrrhizic acid, the bottom row in FIG. 5).

For the BLM-induced scleroderma model mice, mRNA of Snail1, which induces Endo-MT, was measured. As a result, it was found that the mRNA level of Snail1 was significantly decreased by the administration of glycyrrhizic acid.

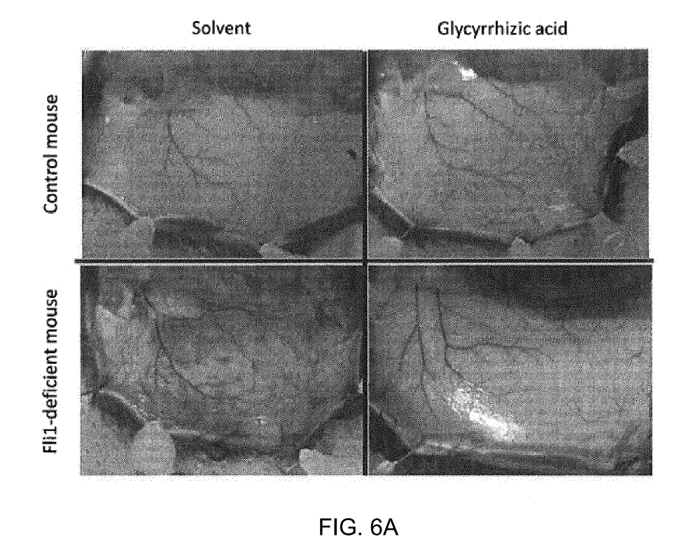

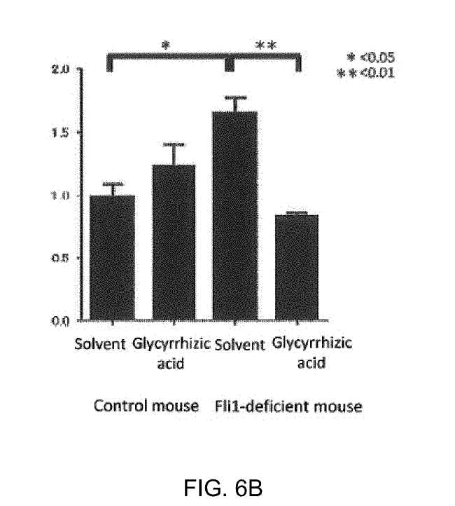

Further, vascular endothelial cell-specific Fli1 knockout mice were prepared as model mice for angiopathy in scleroderma, and extravasation of Evans blue was studied. As a result, extravasation of Evans blue, which had been increased by the Fli1 knockout, was significantly decreased by the administration of glycyrrhizic acid (FIGS. 6, A and B).

From these results, it was suggested that glycyrrhizic acid improves stability of blood vessels. The improvement of stability was suggested to be due to suppression of endothelial-to-mesenchymal transition.

### INDUSTRIAL APPLICABILITY

Administration of the prophylactic and/or therapeutic agent for scleroderma of the present invention enables prevention and/or treatment of scleroderma. The glycyrrhetinic acids and/or salts thereof contained in the agent of the present invention is/are a major pharmacologically active component(s) of glycyrrhiza, which has been used in traditional Chinese medicine from ancient times, and safety of the component(s) has been widely recognized. Therefore, the agent safely enables prevention and/or treatment of scleroderma. The glycyrrhetinic acids and/or salts thereof can also be used as a Th2 promotion inhibitor or a vascular stabilizing agent.

1.-6. (canceled)

7. A method for prevention and/or treatment of scleroderma, comprising:
administering glycyrrhetinic acids and/or salts thereof to a subject in need thereof.

8. The method according to claim 7, wherein said glycyrrhetinic acids comprise glycyrrhizic acid.

9. The method according to claim 7, wherein said salts of the glycyrrhetinic acids comprise an ammonium salt or an alkali metal salt.

10. The method according to claim 7, wherein said scleroderma comprises systemic scleroderma.

11. A method for inhibition of Th2 promotion, comprising:
administering glycyrrhetinic acids and/or salts thereof to a subject in need thereof.

12. A method for stabilizing blood vessels, comprising:
administering glycyrrhetinic acids and/or salts thereof to a subject in need thereof.
